(19) United States
(12) Patent Application Publication
(10) Pub. No.: US 2026/0115014 A1
(43) Pub. Date: Apr. 30, 2026

(54) THREE-AXIS ACCELEROMETER CORRECTION OF HEIGHT-BASED ERROR IN IMPLANTABLE MEDICAL DEVICE PRESSURE MEASUREMENT

(71) Applicant: Boston Scientific Scimed, Inc., Maple Grove, MN (US)

(72) Inventors: Sean Thomas Horan, Minneapolis, MN (US); Jessica Williams, Marlborough, MA (US); Natalie Ann Borgos, Roseville, MN (US); John D. Hatlestad, Maplewood, MN (US)

(21) Appl. No.: 19/367,246

(22) Filed: Oct. 23, 2025

Related U.S. Application Data
(60) Provisional application No. 63/713,363, filed on Oct. 29, 2024.

(51) Int. Cl. A61F 2/48; A61F 2/00; G01P 15/18

(52) U.S. Cl. CPC A61F 2/482; A61F 2/004; A61F 2/484; A61F 2250/0013; A61F 2250/0096; G01P 15/18

(57) ABSTRACT

An implantable medical device that includes an inflatable member, a fluid reservoir, a first pressure sensor connected to the fluid reservoir, a second pressure sensor connected to the inflatable member, an accelerometer configured to detect an acceleration of the implantable medical device along three orthogonal directions, and an electronic pump device. The electronic pump device includes a controller that is configured to execute operations that include receiving a first signal with pressure readings from the first pressure sensor and computing an ambient pressure value based on the pressure readings. The operations further include detecting an inflation pressure of the inflatable member using the second pressure sensor, receiving a second signal with acceleration data from the accelerometer, computing a gauge pressure based on the ambient pressure, the acceleration data, and the inflation pressure, and controlling inflation or deflation of the inflatable member based on the gauge pressure.

### CROSS REFERENCE TO RELATED APPLICATION

This application claims priority to U.S. Provisional Patent Application No. 63/713,363, filed on Oct. 29, 2024, entitled “THREE-AXIS ACCELEROMETER CORRECTION OF HEIGHT-BASED ERROR IN IMPLANTABLE MEDICAL DEVICE PRESSURE MEASUREMENT”, the disclosure of which is incorporated by reference herein in its entirety.

### TECHNICAL FIELD

This disclosure relates generally to an implantable medical device and, in particular, to using a three-axis accelerometer to correction height-based errors in pressure measurements within the implantable medical device.

### BACKGROUND

Some implantable medical devices have a pressure sensor to measure the pressure of an inflatable member. However, according to some conventional techniques, it may be difficult to measure (e.g., accurately measure) ambient pressure, which may be used to control inflation and/or deflation of an implantable member by an electronic pump device.

### SUMMARY

In some aspects, the techniques described herein relate to an implantable medical device that includes an inflatable member, a fluid reservoir, a first pressure sensor connected to the fluid reservoir, a second pressure sensor connected to the inflatable member, an accelerometer configured to detect an acceleration of the implantable medical device along three orthogonal directions, and an electronic pump device. The electronic pump device includes a controller that is configured to execute operations that include receiving a first signal with pressure readings from the first pressure sensor and computing an ambient pressure value based on the pressure readings. The operations further include detecting an inflation pressure of the inflatable member using the second pressure sensor, receiving a second signal with acceleration data from the accelerometer, computing a gauge pressure based on the ambient pressure, the acceleration data, and the inflation pressure, and controlling inflation or deflation of the inflatable member based on the gauge pressure.

Implementations can include one or more of the following features, alone or in any combination with each other.

For example, the acceleration data can indicate an orientation of the implantable medical device in space.

In another example, computing the gauge pressure based on the ambient pressure, the acceleration data, and the inflation pressure can include offsetting the gauge pressure from the ambient pressure by an amount based on the indicated orientation.

In another example, computing the gauge pressure based on the ambient pressure, the acceleration data, and the inflation pressure can include offsetting the gauge pressure from the ambient pressure by an amount based on the indicated orientation and a predetermined value indicating a distance (e.g., greater than 20 cm) between the reservoir and the inflatable member.

In another example, the operations can further include obtaining, from a memory device, a correction value relating to a posture of a patient within whom the implantable medical device is implanted and generating an updated ambient pressure based on the ambient pressure value and the correction value.

In another example, the operations can further include applying a trained machine learning model to the received acceleration data to determine a correction value relating to a posture of a patient within whom the implantable medical device is implanted and generating an updated ambient pressure based on the ambient pressure value and the correction value.

In another example, the operations can further include determining an activity state of a patient in whom the implantable medical device is implanted, based on the received acceleration data, generating a correction value based on the determined activity state, and generating an updated ambient pressure based on the ambient pressure value and the correction value.

In another example, the operations can further include applying a trained machine learning model to the received acceleration data to determine the activity state of the patient in whom the implantable medical device is implanted.

In another example, the operations can further include detecting a triggering event, and, in response to detecting the triggering event, activating the first pressure sensor to receive the pressure readings during a time interval, identifying a portion of the pressure readings from the time interval, where the identified pressure readings are within a percentile range, and computing the ambient pressure value based on the portion of the pressure readings.

In some aspects, the techniques described herein relate to a method of operating an implantable fluid-operated medical device that includes an inflatable member and a fluid reservoir, where the method includes receiving pressure readings of a pressure of fluid in the fluid reservoir, computing an ambient pressure value based on the pressure readings, detecting an inflation pressure of the inflatable member, receiving three-dimensional acceleration data from an accelerometer in the implantable medical device, computing a gauge pressure based on the ambient pressure, the acceleration data, and the inflation pressure, and controlling a flow of fluid between the reservoir and the inflatable member, based at least in part on the gauge pressure, to achieve a predetermined inflation or deflation of the inflatable member.

Implementations can include one or more of the following features, alone or in any combination with each other.

For example, the acceleration data can indicate an orientation of the implantable medical device in space.

In another example, computing the gauge pressure based on the ambient pressure, the acceleration data, and the inflation pressure can include offsetting the gauge pressure from the ambient pressure by an amount based on the indicated orientation.

In another example, computing the gauge pressure based on the ambient pressure, the acceleration data, and the inflation pressure can include offsetting the gauge pressure from the ambient pressure by an amount based on the indicated orientation and a predetermined value indicating a distance (e.g., greater than 20 cm) between the reservoir and the inflatable member.

In another example, the method can further include obtaining a correction value relating to a posture of a patient within whom the implantable medical device is implanted and generating an updated ambient pressure based on the ambient pressure value and the correction value.

In another example, the method can further include applying a trained machine learning model to the received acceleration data to determine a correction value relating to a posture of a patient within whom the implantable medical device is implanted and generating an updated ambient pressure based on the ambient pressure value and the correction value.

In another example, the method can further include determining an activity state of a patient in whom the implantable medical device is implanted, based on the received acceleration data, generating a correction value based on the determined activity state, and generating an updated ambient pressure based on the ambient pressure value and the correction value.

In another example, the method can further include applying a trained machine learning model to the received acceleration data to determine the activity state of the patient in whom the implantable medical device is implanted.

In another example, the method can further include detecting a triggering event, in response to detecting the triggering event, activating a first pressure sensor to receive the pressure readings during a time interval, identifying a portion of the pressure readings from the time interval, where the identified pressure readings are within a percentile range, and computing the ambient pressure value based on the portion of the pressure readings.

### BRIEF DESCRIPTION OF THE DRAWINGS

FIG. 1A illustrates an implantable medical device with an electronic pump device for computing an ambient pressure using a pressure sensor connected to a fluid reservoir according to an aspect.

FIG. 1B illustrates an example of a controller for computing an ambient pressure according to an aspect.

FIG. 1C illustrates a histogram of a plurality of pressure readings collected by a pressure sensor according to an aspect.

FIG. 1D illustrates a histogram of a portion of the plurality of pressure readings that fall in a certain percentile range according to an aspect.

FIG. 1E illustrates pressure readings across a plurality of time intervals according to an aspect.

FIG. 2 illustrates an example of a controller that uses a shorter sampling period to determine whether to activate a longer sampling period for computing an ambient pressure value according to an aspect.

FIG. 3 illustrates an example of a controller that retrieves a correction value that is used to adjust an ambient pressure according to an aspect.

FIG. 4A illustrates an example of a controller that generates a correction value during a calibration process according to an aspect.

FIG. 4B is a schematic diagram of a patient in whom an implantable medical device that includes a fluid reservoir, an inflatable member, and an electronic pump device is implanted.

FIG. 5 illustrates an example of a controller with a decimator for downsampling a signal generated by a pressure sensor according to an aspect.

FIG. 6 illustrates a perspective of an inflatable penile prosthesis according to an aspect.

FIG. 7 illustrates an example of an artificial urinary sphincter device according to an aspect.

FIG. 8 illustrates a flowchart depicting example operations of operating an implantable fluid-operated medical device that includes an inflatable member and a fluid reservoir.

### DETAILED DESCRIPTION

This disclosure relates to an implantable medical device configured to detect ambient pressure inside of a body of a patient using reservoir pressure. The ambient pressure may be used to control inflation and/or deflation of an inflatable member. The ambient pressure may be the pressure of the tissues and/or fluids that surround the inflatable medical device, which may be influenced by body position, fluid levels, and/or muscle activity. The implantable medical device includes an inflatable member, a fluid reservoir, and an electronic pump device that transfers fluid between the fluid reservoir and the inflatable member. In some examples, the implantable medical device includes an inflatable penile prosthesis with one or more inflatable cylinders. In some examples, the implantable medical device includes a urinary control device with an inflatable cuff. The electronic pump device may automatically transfer fluid between the inflatable member and the fluid reservoir.

The implantable medical device includes a first pressure sensor connected to the fluid reservoir and a second pressure sensor connected to the inflatable member. The first pressure sensor may detect pressure (e.g., reservoir pressure) in the fluid reservoir. The second pressure sensor may detect pressure (e.g., inflation pressure) in the inflatable member. The electronic pump device includes a controller configured to detect ambient pressure using the first pressure sensor and detect inflation pressure using the second pressure sensor. The controller may compute a gauge pressure using the ambient pressure and the inflation pressure, where the electronic pump device can control inflation and/or deflation of the inflatable member using the gauge pressure.

The controller is configured to determine and update the ambient pressure based on a signal with pressure readings generated by the first pressure sensor. The pressure readings may indicate a pressure level over a time interval. For example, the controller may activate the first pressure sensor to obtain a signal with pressure readings during a time interval (e.g., a set or predetermined period of time) (e.g., a sampling period). The time interval may be thirty seconds, one minute, two minutes, or three minutes, or generally any set length of time. The first pressure sensor may generate the signal with the pressure readings according to a sampling rate. Each pressure reading includes a pressure value, and, in some examples, a timestamp. The controller activates the first pressure sensor to obtain the signal with the pressure readings during the time interval in response to the detection of a triggering event. In some examples, the triggering event may be expiration of a timer or a specified time in a sampling schedule (e.g., one or more times a day, one or more times a week, etc.).

The controller processes the signal to identify a portion of the pressure readings that are within a certain percentile range of the pressure readings during the time interval. The percentile range is defined by a first percentile threshold (e.g., a low percentile threshold) and a second percentile threshold (e.g., a high percentile threshold). In some examples, the percentile range includes or is less than the 50^{th} percentile of the pressure readings. In some examples, the percentile range is the 5^{th} to 50^{th} percentile. In some examples, the percentile range is the 5^{th} to 40^{th} percentile. In some examples, the percentile range is the 5^{th} to 20^{th} percentile. In some examples, the controller uses a lower percentile range to filter out pressure readings caused by short-term drops in pressure and bias due to raised intrabdominal pressure (IAP). In some examples, the controller ranks the pressure readings from the signal by the magnitude of the pressure values and selects the pressure readings within the specified percentile range.

The controller computes an ambient pressure value for the time interval based on the portion of the pressure readings from the signal that are within the percentile range. In some examples, the controller selects a pressure value from one of the pressure readings from the signal that are within the percentile range. In some examples, the controller selects a pressure reading with a minimum pressure value among the portion of the pressure readings that are within the percentile range. In some examples, the controller computes an average pressure value from the portion of the pressure readings that are within the percentile range.

In some examples, the controller activates the first pressure sensor to generate a signal with pressure readings according to a first sampling rate (e.g., a higher sampling rate). In some examples, the controller includes a decimator configured to generate a downsampled signal from the signal, where the downsampled signal includes pressure readings according to a second sampling rate (e.g., a lower sampling rate). A decimator may be a digital signal processing (DSP) filter configured to reduce the sampling rate of a discrete-time signal. Then, the controller may identify a portion of the pressure readings from the downsampled signal that are within a certain percentile range of the pressure readings and use those pressure readings to compute an ambient pressure value (e.g., select a pressure reading with a lowest pressure value among the portion that fall within the percentile range).

In some examples, the controller includes logic for determining whether to accept or reject an ambient pressure value computed by the controller for a current time interval. For example, the controller may compute a threshold deviation from a median using the portion of the pressure readings (e.g., the pressure readings that fall within the percentile range). In some examples, the threshold deviation is an absolute deviation. In some examples, the threshold deviation is a standard deviation. In some examples, the threshold deviation is a variance (e.g., averaged squared deviation from the mean). In some examples, the threshold deviation is peak width. The controller may determine whether a deviation of the ambient pressure value from the median is equal to or greater than the threshold deviation. In response to the deviation of the ambient pressure value from the median being equal to or greater than the threshold deviation, the controller may discard (e.g., reject) the ambient pressure value. If rejected, the controller may wait until the next sampling period (e.g., in response to detection of a subsequent triggering event) to re-collect pressure readings to re-compute the ambient pressure value. If rejected, the ambient pressure is not updated (e.g., the controller may continue to use the previous ambient pressure value from a previous time interval for computation of a gauge pressure). In response to the deviation of the ambient pressure value from the median being less than the threshold deviation, the controller may accept the ambient pressure value.

In some examples, the controller may use frequent shorter measurements to detect ambient pressure changes, and, if a large ambient pressure change is detected, the controller may trigger a longer measurement. In some examples, the use of frequent shorter measurements may save the battery life of the pump's battery. The controller periodically initiates the first pressure sensor to generate pressure readings (e.g., first pressure readings) in a first time interval (e.g., a shorter time interval), and, if there is a relatively large difference between the ambient pressure value and a previous ambient pressure value, the controller may activate (e.g., immediately activate) the first pressure sensor to generate pressure readings (e.g., second pressure readings) in a second time interval (e.g., a longer time interval).

For example, the controller may activate the first pressure sensor to generate first pressure readings during a shorter time interval. In response to a difference between an ambient pressure value for the current (e.g. shorter) time interval and an ambient pressure value for a previous time interval being equal to or greater than a threshold level, the controller may activate (e.g., immediately activate) the first pressure sensor to generate second pressure readings in a longer time interval. In response to a difference between an ambient pressure value for the current (e.g. shorter) time interval and an ambient pressure value for a previous time interval being less than a threshold level, the controller may wait for the next measurement period (e.g., the next triggering event). The subsequent triggering event may be the activation of another shorter sampling period after a period of time, or the activation of a longer sampling period.

In some examples, the controller can obtain a correction value from a memory device of the electronic pump device and uses the correction value to adjust the ambient pressure. The correction value may be a correction factor that adjusts the ambient pressure to account for the user's body. For example, a bias may exist between the fluid reservoir and ambient pressure that may be unique to the patient, which may depend on the implant's location and implementation with the patient, and/or on the patient's body composition. In some examples, the correction value is a user-specific value (or user class-specific value). In some examples, the controller may compute the correction value during a calibration process of the implantable medical device. For example, the electronic pump device may include an accelerometer that can measure acceleration in three orthogonal directions. During a calibration process, the user may be instructed to position their body in a plurality of positions, where the controller receives acceleration data from the accelerometer. The controller may generate the correction value based on the acceleration data. The use of the accelerometer may assist with determining the height differential between the fluid reservoir and the inflatable member and/or assist with correcting the changes to the reservoir pressure caused by position change and movement.

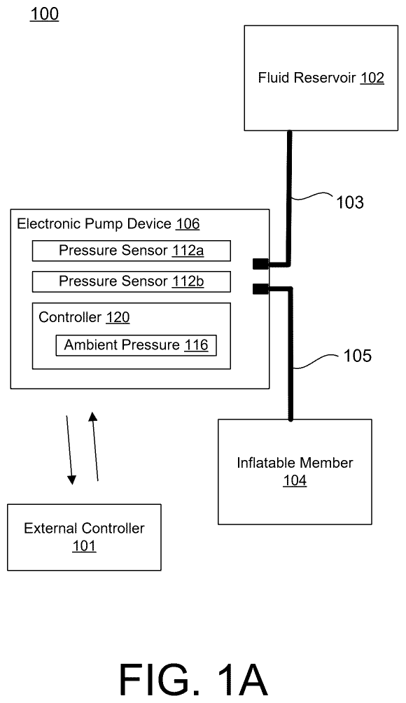

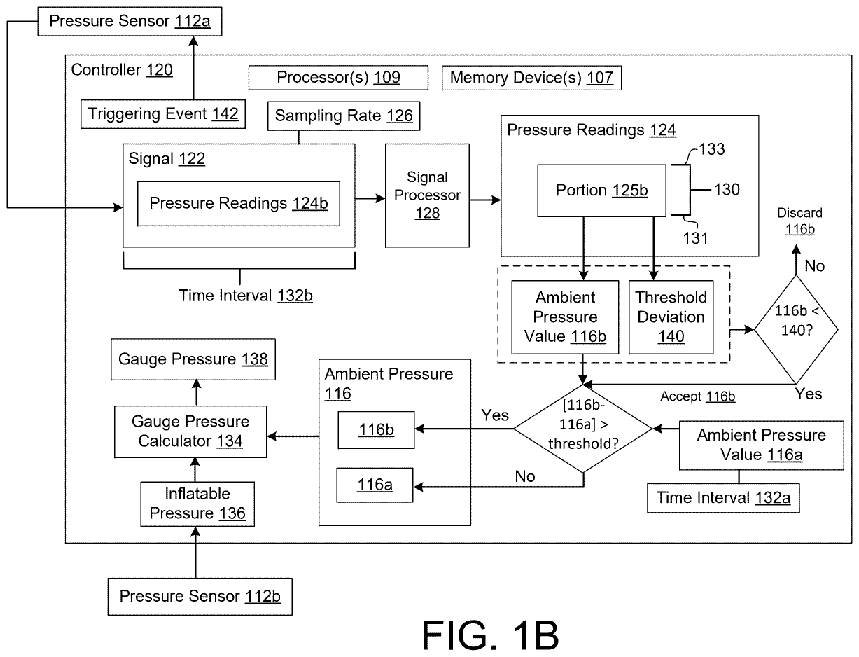

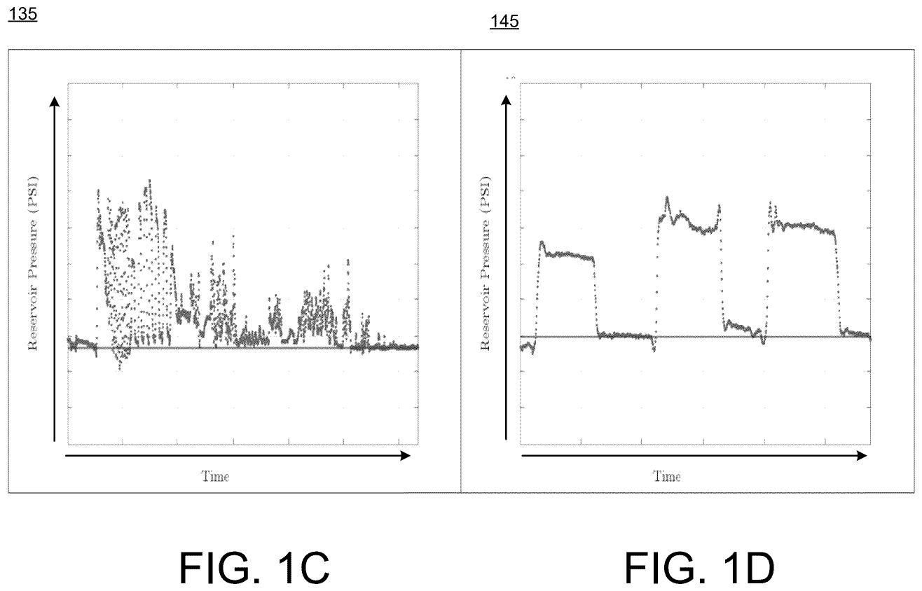

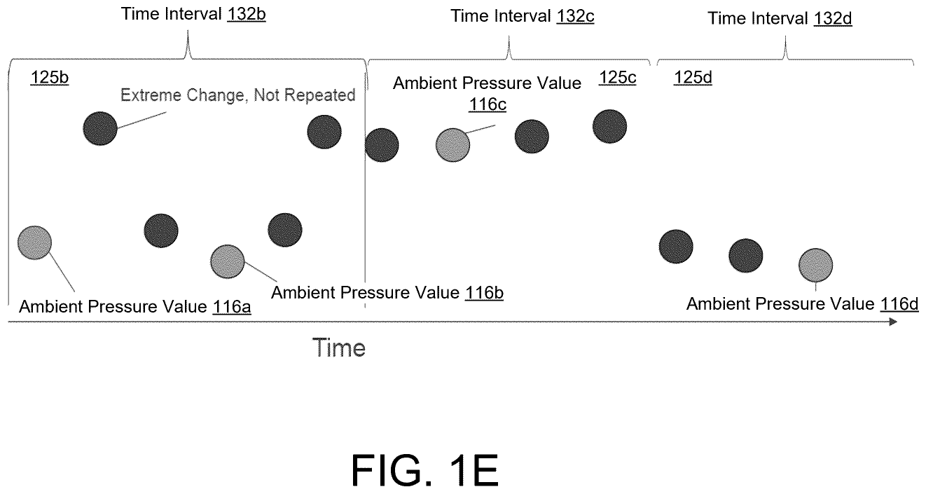

FIGS. 1A to 1E illustrate an implantable medical device 100 that monitors and computes ambient pressure 116 inside of a body of a patient using a pressure sensor 112a connected to a fluid reservoir 102. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100.

As shown in FIG. 1A, the implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile prosthetic with one or more inflatable cylinders that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102. In some examples, the pressure in the fluid reservoir 102 is greater than the pressure in the inflatable member 104 (e.g., even when the inflatable member 104 is at its target or maximum pressure). In some examples, the pressure in the fluid reservoir 102 is always greater than the pressure in the inflatable member 104.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be fluidically connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 includes pumps, valves, a battery, and electronic circuitry. The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device 101 to inflate or deflate the inflatable member 104.

The electronic pump device 106 includes one or more pressure sensors. The electronic pump device 106 may include a pressure sensor 112a connected to the fluid reservoir 102. The pressure sensor 112a is used for measuring a pressure (e.g., reservoir pressure) of the fluid reservoir 102. The electronic pump device 106 includes a pressure sensor 112b connected to the inflatable member 104. The pressure sensor 112b is used for measuring a pressure (e.g., inflation pressure) of the inflatable member 104.

The electronic pump device 106 includes a controller 120 configured to monitor, compute, and/or update an ambient pressure 116. The ambient pressure 116 may be used to control inflation and/or deflation of an inflatable member 104. The ambient pressure 116 may be the pressure of the tissues and/or fluids that surround the implantable medical device 100, which may be influenced by body position, fluid levels, and/or muscle activity.

In some examples, the controller 120 determines the ambient pressure 116 using the pressure sensor 112a (e.g., a pressure sensor connected to the fluid reservoir 102). In some examples, cylinder pressure sensors (e.g., pressure sensor 112b) may be biased above or below ambient pressure 116 by movement, and/or manipulation, etc. However, in some examples, bias on the reservoir sensor may be caused by IAP changes and/or external pressures, which typically increases pressure (e.g., only increases). By using a pressure sensor connected to the fluid reservoir 102 for computing ambient pressure 116, the implantable medical device 100 may be less susceptible to being affected (e.g., significantly affected) by temporary or minor errors or inconsistencies in measurements (e.g., it can handle short-term fluctuations or inaccuracies in data without being significantly impacted).

Referring to FIG. 1B, the controller 120 includes one or more processors 109 and one or more memory devices 107. A memory device 107 may store an ambient pressure 116 that is used to compute a gauge pressure 138, and the controller 120 may periodically update the ambient pressure 116 and store the updated ambient pressure 116 in the memory device 107.

The controller 120 may activate the pressure sensor 112a to generate a signal 122 with pressure readings 124b during a time interval 132b. The time interval 132b may have a predetermined length (e.g., thirty seconds, one minute, two minutes, or three minutes, or generally any set length of time). In some examples, a time interval is referred to as a sampling period or a measurement period. In some examples, the controller 120 may periodically activate the pressure sensor 112a to generate the signal 122 for a respective sampling period and determine whether to update the ambient pressure 116. In some examples, updating the ambient pressure 116 includes replacing an old value with a new value in the memory device 107.

The controller 120 activates the pressure sensor 112a to generate the signal 122 with the pressure readings 124b during the time interval 132b in response to the detection of a triggering event 142. In some examples, the triggering event 142 may be expiration of a timer or achieving a time indicated by a sampling schedule (e.g., one or more times a day, one or more times a week, etc.). The pressure sensor 112a may generate the signal 122 with pressure readings 124b according to a sampling rate 126. In some examples, the sampling rate 126 is between 1-10 Hz. Each pressure reading 124b includes a pressure value, and, in some examples, a timestamp. FIG. 1C is a graph 135 of a plurality of pressure readings 124b collected by a pressure sensor 112a as a function of time. FIG. 1D is another graph 145 of a portion 125b of the plurality of pressure readings 124b collected by the pressure sensor 112a.

The controller 120 includes a signal processor 128 that processes the signal 122 to identify a portion 125b of the pressure readings 124b that are within a certain percentile range 130 of the pressure readings 124b during the time interval 132b. The percentile range 130 is defined by a percentile threshold 131 (e.g., a low percentile threshold) and a percentile threshold 133 (e.g., a high percentile threshold). In some examples, the percentile range includes or is less than 50^{th} percentile of the pressure readings. In some examples, the percentile range is 5^{th} to 50^{th} percentile. In some examples, the percentile range is 5^{th} to 40^{th} percentile. In some examples, the percentile range is 5^{th} to 20^{th} percentile. In some examples, the controller uses a lower percentile range to filter out pressure readings 124b caused by short-term drops in pressure and bias due to raised IAP. In some examples, the controller 120 ranks the pressure readings 124b from the signal 122 by the magnitude of the pressure values and selects the pressure readings 124b within the specified percentile range.

The controller 120 generates an ambient pressure value 116b based on the portion 125b of the pressure readings 124b from the signal 122 that are within the percentile range 130. In some examples, the controller 120 selects a pressure value from one of the pressure readings 124b from the signal 122 that are within the percentile range 130. In some examples, the controller 120 selects a pressure reading 124b with a minimum pressure value among the portion 125b of the pressure readings 124b that are within the percentile range 130. In some examples, the controller 120 computes an average pressure value from the portion 125b of the pressure readings 124 that are within the percentile range 130.

In some examples, the controller 120 includes logic for determining whether to accept or reject the ambient pressure value 116b computed by the controller 120 for a current time interval (e.g., time interval 132b). For example, the controller 120 may compute a threshold deviation 140 using the portion 125b of the pressure readings 124b (e.g., the pressure readings 124b that fall within the percentile range 130).

In some examples, the threshold deviation 140 is an absolute deviation. In some examples, the threshold deviation 140 is a standard deviation. In some examples, the threshold deviation 140 is a variance (e.g., averaged squared deviation from the mean). In some examples, the threshold deviation 140 is peak width.

The controller 120 may determine whether a deviation of the ambient pressure value 116b from the median is equal to or greater than the threshold deviation 140. In response to the deviation of the ambient pressure value 116b from the median being equal to or greater than the threshold deviation 140, the controller 120 may discard (e.g., reject) the ambient pressure value 116b. If rejected, the controller 120 may wait until the next sampling period (e.g., in response to detection of a subsequent triggering event 142) to re-collect pressure readings (e.g., time interval 132c) to re-compute the ambient pressure value (e.g., ambient pressure value 116c). In response to the deviation of the ambient pressure value 116b from the median being less than the threshold deviation 140, the controller 120 may accept the ambient pressure value 116b.

In some examples, when the ambient pressure value 116b is accepted, the controller 120 may compare the ambient pressure value 116b for the current time interval (e.g., time interval 132b) with an ambient pressure value 116a from a previous time interval 132a to determine whether to update the ambient pressure 116 with the ambient pressure value 116b (e.g., a new ambient pressure value) for the current time interval (e.g., time interval 132b) or continue to maintain the previous ambient pressure value 116a. In some examples, if the difference between the ambient pressure value 116b for the current time interval 132b and the ambient pressure value 116a for the previous time interval 132a is equal to or greater than a threshold level, the controller 120 may update the ambient pressure 116 with the ambient pressure value 116b for the current time interval 132b.

If the difference between the ambient pressure value 116a for the current time interval 132b and the ambient pressure value 116a for the previous time interval 132a is less than the threshold level, the controller 120 may use the previous ambient pressure value 116a (e.g., the ambient pressure 116 is not updated). In some examples, if the ambient pressure value 116b for the current time interval 132b is less than the ambient pressure value 116a for the previous time interval 132a, the controller 120 uses the new ambient pressure value (e.g., the ambient pressure value 116b). In some examples, if the ambient pressure value 116b for the current time interval 132b is greater than the previous ambient pressure value 116a by a threshold level, the controller 120 uses the new ambient pressure value (e.g., the ambient pressure value 116b).

The controller 120 detects an inflatable pressure 136 of the inflatable member 104 using the pressure sensor 112b. The controller 120 includes a gauge pressure calculator 134 that computes a gauge pressure 138 using the ambient pressure 116 and the inflatable pressure 136. In some examples, the gauge pressure calculator 134 offsets the inflatable pressure 136 by the ambient pressure 116.

The memory device(s) 107 may store executable instructions that when executed by the processor(s) 109 cause the processor(s) to execute the operations of the controller 120 as discussed herein. In some examples, the memory device(s) 107 include a non-transitory computer-readable medium or computer program product. In some examples, the controller 120 and the pressure sensors (e.g., pressure sensor 112a, pressure sensor 112b) may be stored on a printed circuit board in a housing of the electronic pump device 106. In some examples, the controller 120 is included in a printed circuit board and is attached to a manifold structure that also includes one or more pumps and one or more valves for transferring fluid between the inflatable member 104 and the fluid reservoir 102.

FIG. 1E illustrates pressure readings for three time intervals, e.g., time interval 132b, time interval 132c, and time interval 132d. The time intervals may not be directly adjacent to each other, and a period of time may exist between successive time intervals. In some examples, the length of the time interval 132b, the time interval 132c, and the time interval 132d is the same. In the time interval 132b, a portion 125b of pressure readings are selected that achieve a percentile range 130. The controller 120 determines that an ambient pressure value 116b is the lowest among the portion 125b of the pressure readings in the time interval 132b and updates the ambient pressure 116 with the ambient pressure value 116b. In some examples, the previous ambient pressure value is an ambient pressure value 116a, and, since the ambient pressure value 116b is less than the previous ambient pressure value, the controller 120 updates the ambient pressure 116 in the memory device 107 from the ambient pressure value 116a to the ambient pressure value 116b.

In a subsequent time interval (e.g., time interval 132c), a portion 125c of pressure readings are selected that achieve the percentile range 130. The controller 120 determines that an ambient pressure value 116c is the lowest among the portion 125c of the pressure readings in the time interval 132c and updates the ambient pressure 116 with the ambient pressure value 116c in the memory device 107. In the time interval 132c, a large increase in pressure values is repeatedly observed, and, therefore, the previous ambient value (e.g., ambient pressure value 116b) is discarded, and the lowest pressure value (e.g., the ambient pressure value 116c) in the time interval 132c is selected as the new pressure value for the ambient pressure 116.

In another subsequent time interval (e.g., time interval 132d), a portion 125d of pressure readings are selected that achieve the percentile range 130. The controller 120 determines that an ambient pressure value 116d is the lowest among the portion 125d of the pressure readings in the time interval 132d and updates the ambient pressure 116 with the ambient pressure value 116d in the memory device 107. In the time interval 132d, a decrease in pressure values is repeatedly observed, and, therefore, the previous ambient value (e.g., ambient pressure value 116c) is discarded, and the lowest pressure value (e.g., the ambient pressure value 116d) in the time interval 132d is selected as the new pressure value for the ambient pressure 116.

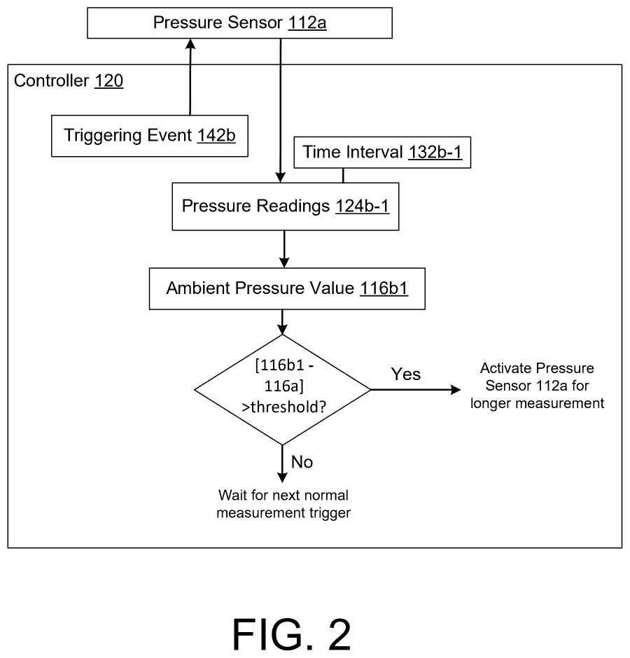

FIG. 2 illustrates an example of a controller 120 that uses a shorter sampling period to determine whether to activate a longer sampling period for computing an ambient pressure value according to an aspect. Referring to FIG. 2, the controller 120 may use frequent shorter measurements to detect ambient pressure changes, and, if a large ambient pressure change is detected, the controller 120 may trigger a longer measurement. The use of frequent shorter measurements may increase the amount of time that a pump's battery can operate before the battery may be required to be recharged.

The controller 120 initiates (e.g., periodically initiates) the pressure sensor 112a to generate pressure readings 124b-1 (e.g., first pressure readings) in a time interval 132b-1. The time interval 132b-1 may have a length that is shorter than the normal time interval (e.g., time interval 132b). In some examples, the time interval 132b-1 may be referred to as a shorter time interval. The controller 120 may compute an ambient pressure value 116b1 using the pressure readings 124b-1. In some examples, the controller 120 may select a pressure reading 124b-1 with a lowest value as the ambient pressure value 116b1.

If there is a relatively large difference between the ambient pressure value 116b1 and a previous ambient pressure value 116a, the controller 120 may activate (e.g., immediately activate) the pressure sensor 112a to generate pressure readings (e.g., pressure readings 124b) (e.g., second pressure readings) in the time interval 132b. In some examples, the time interval 132b has a length that is longer than the time interval 132b-1. For example, if the difference between the ambient pressure value 116b1 and the previous ambient pressure value 116a (e.g., computed in a previous (longer) time interval 132a) is equal to or greater than a threshold level, the controller 120 activates (e.g., immediately activates) the pressure sensor 112a to collect pressure readings 124b during a longer interval (e.g., the time interval 132b).

In response to a difference between the ambient pressure value 116b1 for the current (e.g. shorter) time interval 132b-1 and the ambient pressure value 116a for a previous time interval 132a being detected as less than a threshold level, the controller 120 may wait for the next sampling period (e.g., the next triggering event 142 or 142b). The subsequent triggering event may be the activation of another shorter sampling period (e.g., time interval 132b-1), or the activation of a longer sampling period (e.g., time interval 132b).

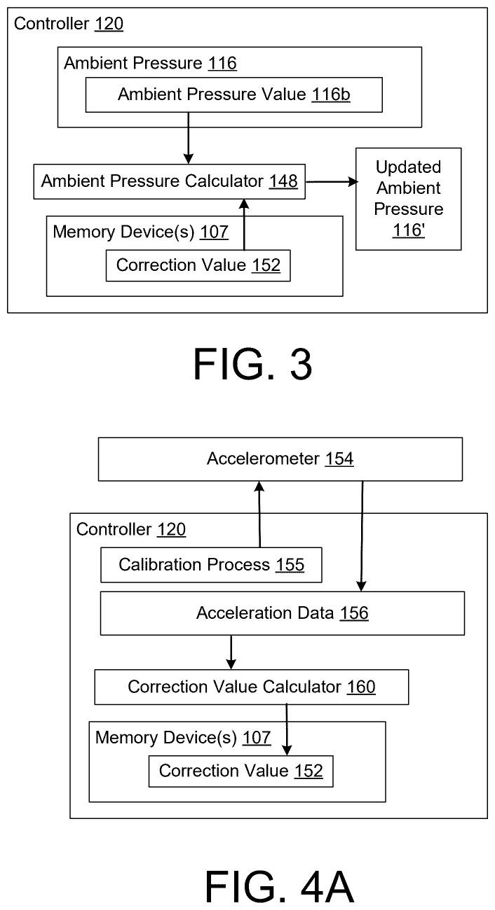

FIG. 3 illustrates an example of a controller 120 that retrieves a correction value 152 that is used to adjust an ambient pressure 116 according to an aspect. Referring to FIG. 3, the controller 120 may obtain a correction value 152 from a memory device 107 of the electronic pump device 106 and use the correction value 152 to adjust the ambient pressure 116.

For example, the controller 120 may include an ambient pressure calculator 148 that retrieves a correction value 152 from the memory device 107. The ambient pressure calculator 148 may also receive the ambient pressure value 116b from the memory device 107 and generate an updated ambient pressure 116′ using the correction value 152. In some examples, the correction value 152 is stored in the memory device 107 before the implantable medical device 100 is implanted in the patient's body. In some examples, the controller 120 stores the correction value 152 after the implantable medical device 100 is implanted in the patient's body. In some examples, the controller 120 receives the correction value 152 from the external device 101. In some examples, the controller 120 generates (e.g., determines) the correction value 152 during a set-up or calibration process of the implantable medical device 100.

The updated ambient pressure 116′ may be an ambient pressure value that accounts for the user's body. For example, the correction value 152 may be a correction factor that adjusts the ambient pressure 116 to account for aspects of the user's body. For example, a bias may exist between the fluid reservoir 102 and ambient pressure 116 that may be unique to the patient's body, and which may depend on the implant's location and orientation in the patient's body and on the patient's body composition. In some examples, the correction value 152 is a user-specific value (or user class-specific value). In some examples, the gauge pressure calculator 134 may use the updated ambient pressure 116′ to compute the gauge pressure 138.

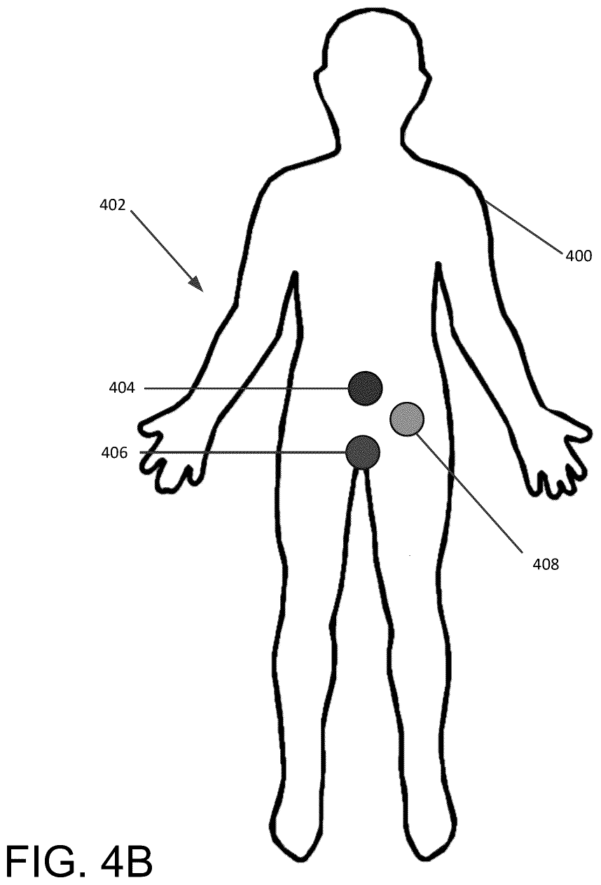

FIG. 4A illustrates an example of a controller 120 that generates a correction value 152 during a calibration process 155 according to an aspect. In some examples, as shown in FIG. 4A, the controller 120 may compute the correction value 152 during a calibration process 155 of the implantable medical device 100. In some examples, the electronic pump device 106 may include an accelerometer 154 configured to determine an acceleration of the electronic pump device 106 along an x-axis, y-axis, and z-axis. In some examples, in response to initiation of the calibration process 155, the controller 120 may activate the accelerometer 154 to obtain the three-dimensional acceleration data 156 with information about the acceleration in an x-axis, a y-axis, and a z-axis.

Acceleration data 156 received from the accelerometer 154 can be used to determine a correction value 152 or to calibrate or otherwise adjust an ambient pressure 116 that is measured based on pressure readings 124b received from the pressure sensor 112a that is fluidically connected to the reservoir, so that the calibrated/adjusted ambient pressure can be used to determine a gauge pressure 138 for the inflatable member 104.

FIG. 4B is a schematic diagram of a patient 400 in whom an implantable medical device 402 that includes a fluid reservoir 404, an inflatable member 406, and an electronic pump device 408 is implanted. When the reservoir 404 and the inflatable member 406 are located at different heights and when they are fluidically connected to each other, for example, through the electronic pump device 408, the static pressures of the fluid in the reservoir 404 and the inflatable member 406 will be different. For example, when the reservoir 404 is 30 cm higher than the inflatable member 406 and the system is filled with a liquid having a density similar to that of water, then the static fluid pressure in the inflatable member can be about 0.4 PSI higher than the static pressure fluid pressure in the reservoir 404. Therefore, when pressure readings received from a pressure sensor connected to the fluid reservoir 404 are used to determine an ambient pressure for the implantable medical device 402 in general, a correction factor can be applied to that determined pressure so that the corrected pressure can be used to accurately determine a gauge pressure of the fluid within the inflatable member 406, where the correction factor is based on the difference in height between the fluid reservoir 404 and the inflatable member 406.

In an implementation, when the implantable medical device 402 is implanted within the patient 400, the relative position of the reservoir 404 with respect to the inflatable member 406 can be measured. The relative position can be measured in three dimensions. From the relative position between the reservoir 404 and the inflatable member 406, a standing height differential between the reservoir 404 and the inflatable member 406 can be determined. In some implementations, the standing height differential can be the same as a supine lateral differential, which can be measured while the patient is lying flat on an operating table. Furthermore, based on the three-dimensional relative position between the reservoir 404 and the inflatable member 406, a maximum height differential between the reservoir and the inflatable member 406 can be determined, where the maximum height differential, in some cases, can be greater than the standing height differential. For example, when the patient 400 is standing vertically erect, the longitudinal axis of the patient 400 be aligned with to the z-axis, the anteroposterior axis of the patient 400 can be aligned with the y-axis, and the horizontal (or left/right) axis of the patient can be aligned with the x-axis, but if the y-and/or x-axis locations of the reservoir 404 and the inflatable member 406 are not identical, then the maximum height differential can be larger than the standing height differential. In other words, the vertical (e.g., z-axis) distance between the reservoir 404 and the inflatable member 406 can be greater than the standing height differential when the patient is oriented at an angle that is different than the vertically erect direction in which the longitudinal axis of the patient 400 is aligned with the z-axis. In some implementations, the distance between the reservoir 404 and the inflatable member 406 can be greater than 20 cm.

As the patient 400 moves about, the relative heights of the reservoir 404 and the inflatable member 406 can change. For example, when the patient 400 is lying down, the height differential between the reservoir 404 and the inflatable member can be much smaller than the maximum height differential, for example, close to zero. In another example, when the patient 400 is in a reclined position, the height differential between the reservoir 404 and the inflatable member can be smaller than the maximum height differential but greater than zero.

The accelerometer 154, which can be located within the housing of the electronic pump device 408, can measure gravitational acceleration values along the x-, y-, and z-axes, which can be used to determine an orientation of the accelerometer. Because the accelerometer 154 is located proximate to the reservoir 404 and the inflatable member 406 the orientation of the accelerometer can be used to determine the relative orientation of the reservoir and the inflatable member. Then, the determined relative orientation of the reservoir 404 and the inflatable member 406, along with the maximum height differential between the reservoir and the inflatable member, can be used to determine an actual height differential between the reservoir in the inflatable member. In a simplified example, when the maximum height differential (hₒ) between the reservoir 404 and the inflatable member 406 occurs when the patient 400 is standing vertically erect, then, when the patient stands at an angle (θ) with respect to the vertical direction, the actual height difference (h) between the reservoir and the inflatable member is h=hₒcosθ.

Thus, the acceleration data 156 received from the accelerometer 154 can be used to determine an orientation of the accelerometer 154 and the correction value calculator 160 can calculate a correction value 152 to be applied to the ambient pressure value that is based on pressure readings from the pressure sensor that is fluidically connected to the reservoir 404. For example, the correction value 152 can be equal to, or proportional to, an actual height difference between the reservoir 404 and the inflatable member 406 multiplied by the specific gravity of the fluid in the implantable medical device 402. Referring again to FIG. 1B, the gauge pressure 138 can be determined based on the application of the correction value 152 to the ambient pressure 116. For example, the correction value 152 can be added to the determined ambient pressure 116.

In some implementations, during a calibration process 155, the patient 400 may be instructed to position their body in a plurality of positions, where the controller 120 receives acceleration data 156 about the plurality of positions from the accelerometer 154. The controller 120 may include a correction value calculator 160 that generates correction values 152 based on the acceleration data 156. The correction value calculator can store 160 the correction values 152 in the memory device 107, which is used to adjust the ambient pressure 116. Then, as explained above, the accelerometer 154 may assist with determining the height differential between the fluid reservoir 102 and the inflatable member 104 and/or assist with correcting the changes to the reservoir pressure caused by position change and movement.

Furthermore, in some implementations, a combination of pressure readings received from the pressure sensor connected to the reservoir 404 and acceleration data 156 received from the accelerometer 154 can be used to determine a gauge pressure 138 to applied to the inflatable member 406 not only based on changes in static orientation of the patient 400 but also based on the posture of the patient as well as based on dynamic changes of the patient's position, movement, and posture.

For example, when the patient 400 is lying down on a flat surface, the orientation of the accelerometer 154 may be the same when the patient's legs are extended flat along the flat surface, when the patient's legs are bent at the knees with the patient's feet on the flat surface, and when the patient's legs are raised with the patient's feet off of the flat surface. However, the IAP within the body of the patient can differ in each of these positions, and therefore a different pressure on the inflatable member 406 may be needed in each of the different positions to achieve a particular therapeutic result. To address these different situations, a machine learning model can be trained based on pressure readings from a pressure sensor connected to the reservoir 404 and based on accelerometer data 156 received from the accelerometer 154, where the data are received when patients assume different positions, to recognize when the patient's body is in different positions and postures. Then, the trained machine learning model can receive inputs of accelerometer data and pressure sensor data and, based on the received data, can determine if the patient's body is in position and/or posture that has been classified during the training of the machine learning model. When a classified position and/or posture is recognized by the machine learning model, the electronic pump device 408 can be controlled to generate a desired pressure in the inflatable member 406.

In another example, in which the inflatable member 406 is an inflatable cuff member configured to restrict the flow of urine through the urethra of the patient 400, a higher gauge pressure in the inflatable member may be needed to restrict the flow of urine when the patient is very active (e.g., running) than when the patient is at rest. The activity state of the patient can be determined based on pressure readings from the pressure sensor connected to the reservoir 404. In some implementations, pressure readings from a pressure sensor connected to the reservoir can be correlated with accelerometer data (which is conventionally used to determine an activity state of a person), while patients are performing different activities (e.g., lying down, sitting, standing, walking, running, jumping, and other known activities). In some implementations, the accelerometer data signature of the different activities may be known, so that pressure sensor data can be correlated with the different activities through the accelerometer data that is received contemporaneously with the accelerometer data. In this manner, pressure sensor data signatures can be used to determine particular activities of a patient in which an implantable medical device 402 is implanted. Then, based on the inferred activity, the electronic pump device 408 can be controlled to generate a desired pressure in the inflatable member 406 that is suitable to achieve a therapeutic result while the patient is performing the inferred activity.

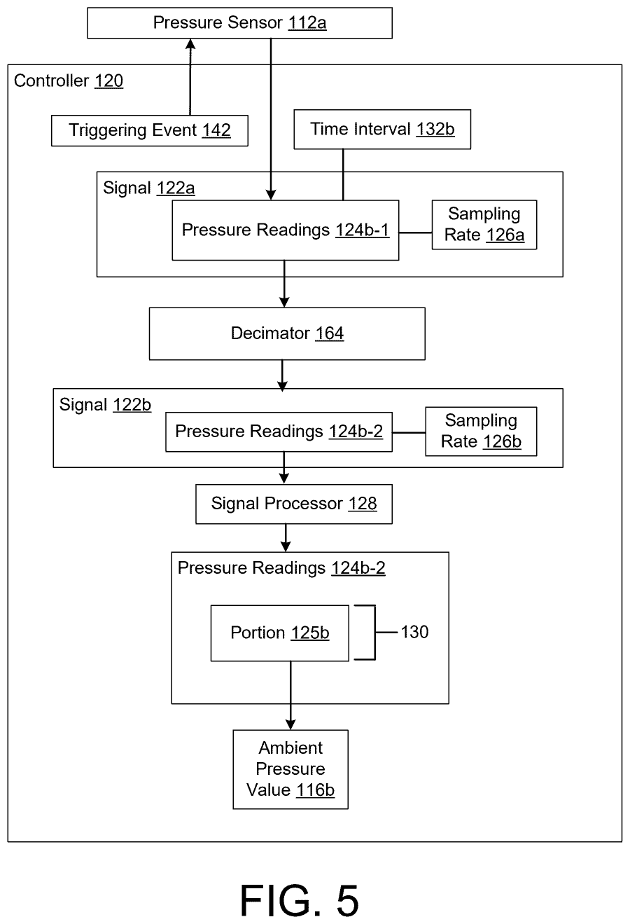

FIG. 5 illustrates an example of a controller 120 with a decimator 164 to downsample a signal 122a generated by a pressure sensor 112a. In response to a triggering event 142, the controller 120 activates the pressure sensor 112a to generate a signal 122a with pressure readings 124b-1 during a time interval 132b according to a sampling rate 126a (e.g., a higher sampling rate). The controller 120 includes a decimator 164 configured to generate a signal 122b (e.g., a downsampled signal) from the signal 122a, where the signal 122b includes pressure readings 124b-2 according to a sampling rate 126b (e.g., a lower sampling rate). The number of pressure readings 124b-2 is less than the number of pressure readings 124b-1. The decimator 164 may be a digital signal processing (DSP) filter used to reduce the sampling rate of a discrete-time signal. Then, the controller 120 may identify a portion 125b of the pressure readings 124b-2 from the signal 122b (e.g., the downsampled signal) that are within a certain percentile range 130 of the pressure readings 124b-2 and may use that portion 125b to generate an ambient pressure value 116b (e.g., select a pressure reading with a lowest pressure value among the portion 125b that fall within the percentile range 130).

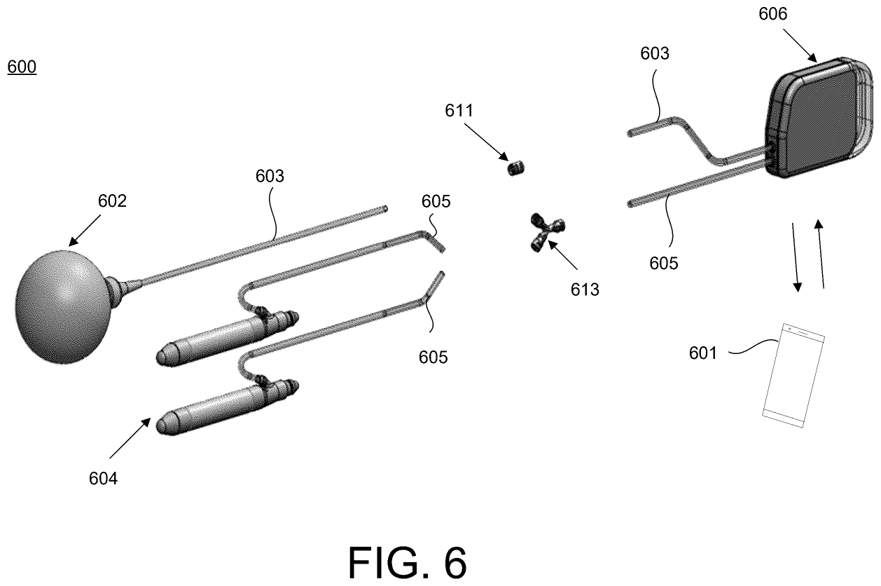

FIG. 6 illustrates a perspective of an inflatable penile prosthesis 600 according to an aspect. The inflatable penile prosthesis 600 may be an example of any of the medical devices discussed herein (e.g., including implantable medical device 100), and, therefore, may include any of the details discussed with reference to the previous figures.

The inflatable penile prosthesis 600 includes an inflatable member 604, a fluid reservoir 602, and an electronic pump device 606. The inflatable member 604 includes a pair of inflatable cylinders. The electronic pump device 606 may be an example of any of the pump devices discussed with reference to the previous figures and may include any of the details discussed herein. The electronic pump device 606 includes fluidics components such as pumps, valves, and/or sensing devices positioned in fluid passageways. The pump device 606 includes components such as, for example, one or more fluid control devices, one or more pressure sensors, and other such components. The electronic pump device 606 includes an electronic control system configured to provide for the transfer of fluid between a reservoir 602 and an inflatable member 604 via the fluidics components.

The electronic pump device 606 may include a controller (e.g., the controller 120) and pressure sensors (e.g., pressure sensor 112a, pressure sensor 112b). In some examples, the controller is included in a printed circuit board that is included in a housing of the electronic pump device 606. Fluidics components and the electronic components of the electronic pump device 106 are included in a housing. In some examples, fluidics components and electronic components in the housing define a manifold (e.g., an electronically controlled fluid manifold) that provides for the electronic control of the flow of fluid between the reservoir 602 and the inflatable member 604. In some examples, the electronic pump device 606 can communicate with an external device 601, via respective communication modules. For example, an application stored in a memory and executed by a processor of the external device 601 may allow the user and/or a physician to operate, view, monitor and alter operation of the inflatable penile prosthesis 600.

The inflatable penile prosthesis 600 includes one or more first tube members 603 that connect a first fluid port of the electronic pump device 606 with the reservoir 602. One or more second tube members 605 connect a second fluid port of the electronic pump device 606 with the inflatable member 604 in the form of the inflatable cylinders. In some examples, the inflatable penile prosthesis 600 includes a connector 611 that is used to connect two tube members 603 together, and a connector 613 that is used to connect two tube members 605 together.

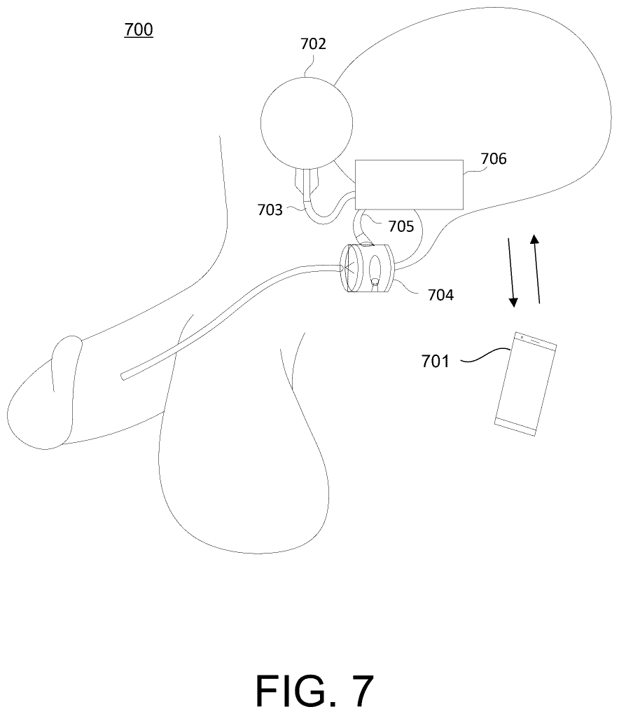

FIG. 7 illustrates a urinary control device 700 having an electronic pump device 706 according to an aspect. The urinary control device 700 may be an example of the implantable medical device 100. In some examples, the urinary control device 700 is an artificial urinary sphincter device. The electronic pump device 706 may include any of the features of the pump devices discussed herein. The urinary control device 700 includes an electronic pump device 706, a fluid reservoir 702, and a cuff 704 (e.g., an inflatable cuff).

The fluid reservoir 702 may be a pressure-regulating inflation balloon or element. The fluid reservoir 702 is in operative fluid communication with the cuff 704 via one or more tube members 703, 705. The fluid reservoir 702 is constructed of polymer material that is capable of elastic deformation to reduce fluid volume within the fluid reservoir 702 and push fluid out of the fluid reservoir 702 and into the cuff 704. However, the material of the fluid reservoir 702 can be biased or include a shape memory construct adapted to generally maintain the fluid reservoir 702 in its expanded state with a relatively constant fluid volume and pressure. In some examples, this constant level of pressure exerted from the fluid reservoir 702 to the cuff 704 will keep the cuff 704 at a desired inflated state when open fluid communication is provided between the fluid reservoir 702 and the cuff 704. In some examples, the fluid reservoir 702 is implanted into the abdominal space.

A user may use an external device 701 to control the urinary control device 700. In some examples, the user may use the external device 701 to inflate or deflate the cuff 704. For example, in response to the user activating an inflation cycle using the external device 701, the external device 701 may transmit a wireless signal to the pump device 706 to initiate the inflation cycle to transfer fluid from the fluid reservoir 702 to the cuff 704 (e.g., by opening an active valve where the pressure in the fluid reservoir 702 causes the fluid to move through the active valve to the cuff 704). In some examples, in response to the user activating a deflation cycle using the external device 701, the external device 701 may transmit a wireless signal to the pump device 706 to initiate the deflation cycle to transfer fluid from the cuff 704 to the fluid reservoir 702.

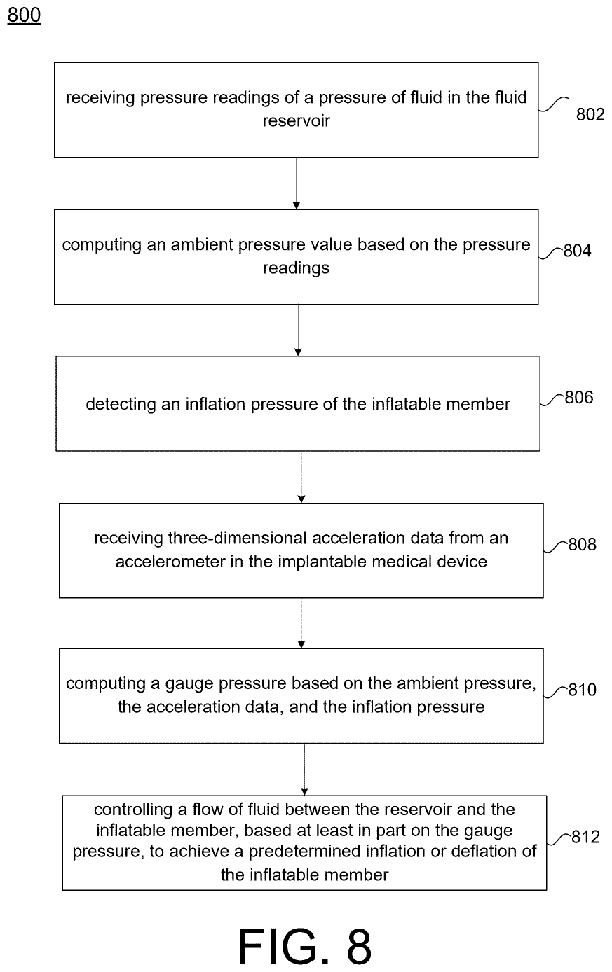

FIG. 8 illustrates a flowchart 800 depicting example operations of operating an implantable fluid-operated medical device that includes an inflatable member and a fluid reservoir according to an aspect. Although the flowchart 800 of FIG. 8 illustrates the operations in sequential order, it will be appreciated that this is merely an example, and that additional or alternative operations may be included. Further, operations of FIG. 8 and related operations may be executed in a different order than that shown, or in a parallel or overlapping fashion.

Operation 802 includes receiving pressure readings of a pressure of fluid in the fluid reservoir. Operation 804 includes computing an ambient pressure value based on the pressure readings. Operation 806 includes detecting an inflation pressure of the inflatable member. Operation 808 includes receiving three-dimensional acceleration data from an accelerometer in the implantable medical device. Operation 810 includes computing a gauge pressure based on the ambient pressure, the acceleration data, and the inflation pressure. Operation 812 includes controlling a flow of fluid between the reservoir and the inflatable member, based at least in part on the gauge pressure, to achieve a predetermined inflation or deflation of the inflatable member.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the implementations described have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir;
a first pressure sensor connected to the fluid reservoir;
a second pressure sensor connected to the inflatable member;
an accelerometer configured to detect an acceleration of the implantable medical device along three orthogonal directions; and
an electronic pump device including a controller configured to execute operations, the operations comprising:
receiving a first signal with pressure readings from the first pressure sensor;
computing an ambient pressure value based on the pressure readings;
detecting an inflation pressure of the inflatable member using the second pressure sensor;
receiving a second signal with acceleration data from the accelerometer;
computing a gauge pressure based on the ambient pressure, the acceleration data, and the inflation pressure; and
controlling inflation or deflation of the inflatable member based on the gauge pressure.

2. The implantable medical device of claim 1, wherein the acceleration data indicates an orientation of the implantable medical device in space.

3. The implantable medical device of claim 2, wherein computing the gauge pressure based on the ambient pressure, the acceleration data, and the inflation pressure includes offsetting the gauge pressure from the ambient pressure by an amount based on the indicated orientation.

4. The implantable medical device of claim 3, wherein computing the gauge pressure based on the ambient pressure, the acceleration data, and the inflation pressure includes offsetting the gauge pressure from the ambient pressure by an amount based on the indicated orientation and a predetermined value indicating a distance between the reservoir and the inflatable member.

5. The implantable medical device of claim 4, wherein the distance is greater than 20 cm.

6. The implantable medical device of claim 1, wherein the operations further comprise:
obtaining, from a memory device, a correction value relating to a posture of a patient within whom the implantable medical device is implanted; and
generating an updated ambient pressure based on the ambient pressure value and the correction value.

7. The implantable medical device of claim 1, wherein the operations further comprise:
applying a trained machine learning model to the received acceleration data to determine a correction value relating to a posture of a patient within whom the implantable medical device is implanted; and
generating an updated ambient pressure based on the ambient pressure value and the correction value.

8. The implantable medical device of claim 1, wherein the operations further comprise:
determining an activity state of a patient in whom the implantable medical device is implanted, based on the received acceleration data;
generating a correction value based on the determined activity state; and
generating an updated ambient pressure based on the ambient pressure value and the correction value.

9. The implantable medical device of claim 8, wherein the operations further comprise:
applying a trained machine learning model to the received acceleration data to determine the activity state of the patient in whom the implantable medical device is implanted.

10. The implantable medical device of claim 1, wherein the operations further comprise:
detecting a triggering event;
in response to detecting the triggering event, activating the first pressure sensor to receive the pressure readings during a time interval;
identifying a portion of the pressure readings from the time interval, where the identified pressure readings are within a percentile range; and
computing the ambient pressure value based on the portion of the pressure readings.

11. A method of operating an implantable fluid-operated medical device that includes an inflatable member and a fluid reservoir, the method comprising:
receiving pressure readings of a pressure of fluid in the fluid reservoir;
computing an ambient pressure value based on the pressure readings;
detecting an inflation pressure of the inflatable member;
receiving three-dimensional acceleration data from an accelerometer in the implantable medical device;
computing a gauge pressure based on the ambient pressure, the acceleration data, and the inflation pressure; and
controlling a flow of fluid between the reservoir and the inflatable member, based at least in part on the gauge pressure, to achieve a predetermined inflation or deflation of the inflatable member.

12. The method of claim 11, wherein the acceleration data indicates an orientation of the implantable medical device in space.

13. The method of claim 12, wherein computing the gauge pressure based on the ambient pressure, the acceleration data, and the inflation pressure includes offsetting the gauge pressure from the ambient pressure by an amount based on the indicated orientation.

14. The method of claim 13, wherein computing the gauge pressure based on the ambient pressure, the acceleration data, and the inflation pressure includes offsetting the gauge pressure from the ambient pressure by an amount based on the indicated orientation and a predetermined value indicating a distance between the reservoir and the inflatable member.

15. The method of claim 14, wherein the distance is greater than 20 cm.

16. The method of claim 11, further comprising:
obtaining a correction value relating to a posture of a patient within whom the implantable medical device is implanted; and
generating an updated ambient pressure based on the ambient pressure value and the correction value.

17. The method of claim 11, further comprising:
applying a trained machine learning model to the received acceleration data to determine a correction value relating to a posture of a patient within whom the implantable medical device is implanted; and
generating an updated ambient pressure based on the ambient pressure value and the correction value.

18. The method of claim 11, further comprising:
determining an activity state of a patient in whom the implantable medical device is implanted, based on the received acceleration data;
generating a correction value based on the determined activity state; and
generating an updated ambient pressure based on the ambient pressure value and the correction value.

19. The method of claim 18, further comprising:
applying a trained machine learning model to the received acceleration data to determine the activity state of the patient in whom the implantable medical device is implanted.

20. The method of claim 11, further comprising:
detecting a triggering event;
in response to detecting the triggering event, activating a first pressure sensor to receive the pressure readings during a time interval;
identifying a portion of the pressure readings from the time interval, wherein the identified pressure readings are within a percentile range; and
computing the ambient pressure value based on the portion of the pressure readings.
